(19) United States
(12) Patent Application Publication
(10) Pub. No.: US 2020/0181550 A1
(43) Pub. Date: Jun. 11, 2020

(54) Method and arrangement for producing biogas

(71) Applicant: Metener Oy, Leppävesi (FI)

(72) Inventors: Erkki KALMARI, Leppävesi (FI); Johanna KALMARI, Leppävesi (FI); Luostarinen JUHA, Leppävesi (FI)

(73) Assignee: Metener Oy, Leppävesi (FI)

(21) Appl. No.: 16/339,381

(22) PCT Filed: Oct. 6, 2017
(86) PCT No.: PCT/FI2017/050707

§ 371 Date: Apr. 4, 2019

(30) Foreign Application Priority Data
Oct. 7, 2016 (FI) 20165756

(51) Int. Cl. C12M 1/107; C02F 11/04; C02F 3/28; C12P 7/06; C12M 1/12; C12M 1/00

(52) U.S. Cl. CPC C12M 21/04; C02F 11/04; C02F 3/2806; C02F 2103/20; C12M 25/00; C12M 29/02; C12P 7/06

(57) ABSTRACT

The invention relates to a method and an arrangement for biogas production. The idea is to use the upper section of recovered percolation fluid for moistening the biomass and return the percolation fluid deliberated from the biomass to the bottom section of the fluid reactor.

### FIELD OF INVENTION

This invention relates to a method and an arrangement for producing biogas.

### BACKGROUND

In a traditional arrangement for biogas production the percolation fluid from the biomass is recirculated to the biomass in a biomass reactor (dry reactor) via a fluid reactor. FI20155428 discloses an improved arrangement and method for biogas production. The basic principle of said disclosure is to moisten the biomass in a bioreactor in phases thereby enhancing stabile biogas production using a single biomass reactor only. One drawback of traditional methods is that also an amount of microbes are transferred to the biomass reactor where e.g. the pH conditions are not suitable for them. pH on the biomass reactor usually kills most of the microbes and the microbial count in the fluid reactor decreases. Recovery of the viable microbial mass requires time (about 3 days). As a result the biogas production is inefficient and the production profile is not stabile. Also the quality of the biogas is decreased and the content on methane may be only about 30 v-percent which inevitably results in problems with recovery and use of the biogas. Thus, there is a continuous need for enhanced conditions for microbes and improved production of biogas.

### OBJECTS AND SUMMARY OF THE INVENTION

An object of this invention is to provide an improved method and arrangement for producing biogas. These are achieved as will be described and claimed below.

The first aspect of the invention is a method for producing biogas. According to the invention the method comprises the steps of:
a) recovering percolation fluid from biomass to a fluid reactor; and
b) recirculating the upper fraction of percolation fluid recovered in step (a) to the upper section of said biomass; and
c) recovering the biogas formed.

The second aspect of the invention is an arrangement for producing biogas. According to the invention the arrangement comprises:
a) a biomass reactor;
b) a fluid reactor; and
c) a fluid collector for recovering percolation fluid; and
d) a device for recirculating the percolation fluid to the biomass; and
e) a device for recovering the biogas;

wherein the device for recirculation the percolation fluid is arranged to circulate the upper fraction of the fluid in the fluid reactor to upper section of the biomass.

### BRIEF DESCRIPTION OF THE FIGURES

FIG. 1 shows one embodiment of an arrangement for producing biogas as described here.

### DETAILED DESCRIPTION OF THE INVENTION

The inventors have now surprisingly found that biogas production can be improved by a new and inventive circulation of percolation fluid. The basic idea is to use the upper section of the percolation fluid (pH typically 7.6 to 7.8) for moistening the biomass and return the percolation fluid recovered from the biomass to the bottom section of the fluid reactor. This ensures that most of the living microbes remain in the fluid reactor where pH and nutritional conditions are optimal. This enhances efficient usage of the organic acids and thereby biogas production. Using the method and/or apparatus here described it is possible to achieve high quality biogas having methane content up to 60 v-% or even more. In addition the gas production profile is stable when compared to the traditional production and in certain amount controllable. Also the delay in biogas production is shortened when compared to a traditional system.

Biogas is a mixture of different gases produced by the breakdown of organic matter in the absence of oxygen. It is a renewable energy source and in many cases exerts a very small carbon footprint. Biogas as discussed here is produced by anaerobic digestion of biodegradable materials. Biogas comprises primarily methane (CH₄) and carbon dioxide (CO₂) and may have small amounts of other components like hydrogen sulphide (H₂S) and ammonium (NH₃). In economically feasible production the methane content of biogas should be at least 40 v-percent.

A method for producing biogas as described here comprises recovering percolation fluid from biomass to a fluid reactor; recirculating the upper fraction of percolation fluid recovered in step (a) to the upper section of said biomass; and recovering the biogas formed. The biomass dry-matter content when introduced into the biomass reactor and before starting fluid circulation from the fluid reactor to the biomass reactor may be within range 18-100 wt-%. During the whole process the biomass in a biomass reactor should have a consistency allowing storing it as a pile.

In this connection an expression “applying the fluid on the upper section of said biomass” covers an embodiment where the fluid is applied on the surface of the biomass or slightly below the surface, e.g. 10 or 15 cm below the surface. The fluid can be applied by spraying or e.g. using perforated ducts. Especially when the method is used in cold climate (such as during winter in Scandinavia) it may be beneficial to submerge the ducts into the upper section of the biomass thereby preventing cooling and even freezing of the percolation fluid.

Expression “bottom section of the fluid reactor” includes bottom of the reactor and the lower fourth or fifth of the fluid reactor.

In this connection an expression “upper fraction of percolation fluid” means essentially the upper third, preferably the upper fourth, of the fluid. The suction site for percolation fluid can be located e.g. 5 to 50 cm, preferably 5 to 30 cm, e.g. 10 cm below the surface of the percolation fluid. An advantage of using the upper fraction of percolation fluid is that most of the microbial mass remains in the fluid reactor.

In more detail the method comprises the steps of introducing biomass in the biomass reactor; and recovering (e.g. using a fluid collector) the percolation fluid deliberated from the biomass from to the bottom of the biomass reactor, leading it to the bottom section of the fluid reactor, recirculating the upper fraction of the percolation fluid in the fluid reactor to the upper section of the biomass located in the biomass reactor and recovering the biogas formed. Biogas is recovered and processed further using methods known within the field. Possible further processes are separation of gases and compression.

Recirculation can be done by introducing a fluid collector to the bottom of the biomass reactor to recover the fluid deliberated from the biomass. The fluid is then lead through a pipe to the bottom section of the fluid reactor. The fresh percolation fluid provides the microbes with nutrients. Then the percolation fluid from the upper section of the fluid reactor is recirculated using a piping to the upper section (including the surface) of the biomass reactor to moisten the biomass. Thus, most of the microbes remain in the fluid reactor where the conditions are suitable.

If the biomass introduced to the reactor is dry, it may be useful to moisten it using water or percolation fluid from another reaction. This will speed up the release of the percolation fluid from the biomass and thereby starting of the biogas production first in the fluid reactor and then in both fluid and biomass reactor.

When the method as described here is used the biogas production can begin within 2 to 5 hours in the fluid reactor when suitable inoculant known within the field is used. One example of suitable inoculant is percolation fluid from earlier biogas production process.

During the first days a lot of saccharides and amino acids and various small peptides are released to the percolation fluid. Later the amount of organic acids in increased in the percolation fluid.

Suitable biomasses for the biogas production include agricultural bio-waste (such as manure, plant material, green waste) and biomass such as grass; food waste, municipal waste, sewage, bio-waste and biomasses from forestry, domestic household, food and feed industry, fishing industry, forest industry and also peat and other natural biomasses. Examples of suitable biomass are grass, silage and straw. Biomass may also be a mixture of different kind of biomasses.

In one embodiment filler material for immobilization of microbes on the bottom of the fluid reactor is introduced. A filler material for immobilization of microbes may be e.g. commercial filler material, plastic net or sections of drainpipe which provide adhesion surface to the microbes and thereby immobilize them to the bottom section of the fluid reactor. Aim of the filler is to enhance maintaining the microbes on the bottom section of the fluid reaction. This ensures that the nutrition conditions are beneficial for the microbes and that loss of microbes in recirculation of fluid recirculated (returned) to the biomass reactor is minimal. Loss of living microbes would compromise the process.

The percolation fluid contains remarkable amount of saccharides and amino acids and various small peptides. According to one embodiment described here the saccharides, amino acids or small peptides, or two or all of them, from the percolation fluid are recovered from the percolation fluid using conventional methods. The recovery may be performed using any separation method known within the art.

According to another embodiment the method comprises fermentation of the percolation fluid recovered from the biomass, recovering the alcohol produced and returning the spent fermentation media to the biogas production, e.g. to the fluid reactor.

In one embodiment the saccharides are fermented to alcohols (such as ethanol) or for example acids (e.g. lactic acid) to be used as a raw material in industrial processes. This would allow using the renewable starting material efficiently and with small further investments. For alcohol production yeast is the most promising fermenting organism. The percolation fluid can be fermented as such or after for example pH adjustment or partial purification. Also simultaneous saccharification and fermentation is usable. Spent fermentation media is advantageously returned to the biogas production. For fermentation the fluid is preferably transferred to a separate fermentation vessel where the conditions can be optimized for the fermentative microbe, such as yeast, filamentous fungi or bacteria.

In one embodiment the amino acids and/or small peptides are used as a material in food, feed or pharmaceutical industry.

Biogas production is not an exothermic reaction but requires temperature of about 35 to 42° C. in order to be efficient. This problem can be overcome by insulation layers around the reactors and e.g. heating the walls of biomass reactor and/or heating the fluid in fluid reactor using various equipment. However, the energy demand of these methods is reasonably high, the heating devices (such as heating elements inside or around the wall or immersed in the fluid) are expensive and the efficiency in transferring heat to the biomass and/or percolation fluid is poor. The present inventors have surprisingly found that efficient production of biogas can be achieved and maintained by heating the percolation fluid before is it recirculated to the biomass reactor.

In one embodiment the method comprises heating the percolation fluid before recirculating it to the biomass. In one embodiment a heat exchanger is used for heating. Heating can take place at any location in the fluid reactor or pipe used to deliver percolation fluid to the biomass reactor or even in the fluid distributor. In one embodiment the percolation fluid is heated to temperature of 34 to 45° C., preferably 35 to 42° C. A further advantage of heating is the possibility to regulate the rate of biogas production. Typically biogas production increases while heating the percolation fluid up to 45° C.

In one embodiment two or more parallel biomass reactors are used in combination of single fluid reactor. This further increases the flexibility of the method allowing using said biomass reactors simultaneously or sequentially thereby optimizing the gas production profile.

An arrangement for producing biogas is also described here. Said arrangement comprises:
a) a biomass reactor;
b) a fluid reactor; and
c) a fluid collector for recovering percolation fluid; and
d) a device for recirculating the percolation fluid to moisten the biomass; and
e) a device for recovering the biogas;

wherein the device for recirculating the percolation fluid is arranged to circulate the upper fraction of the fluid in the fluid reactor to the biomass.

In one embodiment the fluid collector of item c. comprises a piping system for recovering the percolation fluid from the biomass, a pipe and a pump (or pipes and pumps) for leading said fluid to the bottom section of said recovered fluid reactor. The piping system of collector can be for example drainpipe located on the bottom of the biomass reactor.

In one embodiment the device of item d. comprises a piping system (i.e. a pipe or pipes) leading the percolation fluid from the upper section of the fluid in the fluid reactor to the upper section of the biomass reactor and a pump. The piping in the biomass reactor contains a fluid distributor for delivering the fluid onto the biomass. At simplest it is a perforation on the piping located to the biomass reactor. In another embodiment said fluid distributor contains means for controlled moistening of the biomass. Such means can be for example a pump or pumps, a valve or valves or a sprayer or sprayers, alone or in any combination. An example of a device for recirculating the percolation fluid to moisten the biomass in phases and several advantages are disclosed in patent document FI20155428.

The arrangement is essentially gas tight in order to allow recovery of the biogas. The device for recovering the biogas can be any device known within the field. Optionally there is also a device or means for compressing the gas and/or separation of the biogas components from each other at least partially.

In one embodiment the arrangement further comprises filler material for immobilization of microbes to the bottom section of the fluid reactor.

In one embodiment the arrangement comprises also a means for heating the percolation fluid before it is recirculated to the biomass. In one embodiment said means is a heat-exchanger configured to heat the percolation fluid of item d.

In one embodiment there is more than one biomass reactors connected to one fluid reactor. This allows continuous use of the system.

Gas cover which may optionally serve as an insulation layer also is used above the biomass reactor in order to recover biogas formed. In one embodiment of the invention one or more of the pipes (pipings) are equipped with one or more valves. Biogas storage above the fluid reactor can be covered by a separate weather protection, when desired.

In one embodiment biogas from the biomass reactor is lead to the biogas storage above the fluid reactor via a pipe equipped with a gas blower.

### DESCRIPTION OF AN EMBODIMENT WITH REFERENCES TO DRAWINGS

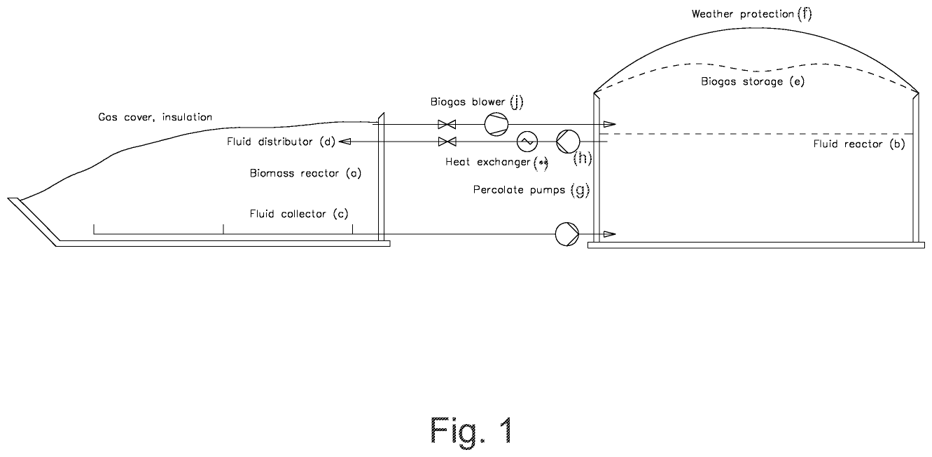

Reference is now made to FIG. 1 which shows one non-restrictive embodiment of the invention. The biomass reactor (a) is loaded with biomass. Loading can be arranged by any conventional means. A fluid collector (c) is located below said biomass. Piping connects the fluid collector to the bottom section of the fluid reactor (b). Usually a pump (g) is needed in order to ensure the flow from the fluid collector to the fluid reactor. Optionally filler material for immobilization of microbes is located to the bottom of the fluid reactor (not shown). The upper section of the fluid in the fluid reactor (b) is connected to the upper section biomass reactor (a) using a pipe. Usually the pipe is equipped with a pump (h) and one or more valves. Fluid distributor (d) can be a simple perforation on the bioreactor section of the pipe but may also comprise valves or e.g. sprinkler system which enhance regulation of the fluid flow and thereby biogas production. In an optional embodiment the pipe leading from fluid reactor to the biomass reactor is also equipped with a heating device (I), such as a heat-exchanger which is used to heat the percolation fluid before it is introduced to the upper section of the biomass. In an alternative embodiment the heating device is located and used in the fluid reactor; e.g. submerged into the fluid or embedded to the walls of said reactor. Recovery of biogas from the biomass reactor is arranged using a pipe equipped with a gas blower (g) to the biogas storage (e) above the fluid reactor. Storage (e) is connected to means for further processing the gas e.g. by compression (not shown).

It is to be understood that the terminology employed herein is for the purpose of description and should not be regarded as limiting. It must be understood, that the embodiments given in the description above are for illustrative purposes only, and that various changes and modifications are possible within the scope of the disclosure.

The features of the invention described here as separate embodiments may also be provided in combination in a single embodiment. Also various features of the described here in the context of the method are usable in connection with the arrangement and vice versa.

1. A method for producing biogas comprising the steps of:
a) recovering percolation fluid from biomass to a fluid reactor b); and
b) recirculating an upper fraction of the percolation fluid recovered in step a) to an upper section of said biomass; and
c) recovering the biogas formed,
wherein the upper fraction of the percolation fluid refers to the upper third of the percolation fluid.

2. The method according to claim 1, further comprising the steps of:
a) introducing the biomass in a biomass reactor; and
b) recovering the percolation fluid from the biomass to the bottom section of the fluid reactor.

3. The method according to claim 1, wherein saccharides, amino acids or small peptides or two or all of them are recovered from the percolation fluid.

4. The method according to claim 1, further comprising:
a) fermenting the percolation fluid recovered from the biomass;
b) recovering ethanol that is thereby produced; and
c) returning spent fermentation media to the biogas production.

5. The method according to claim 1, further comprising heating the percolation fluid before recirculating it to the biomass, preferably using a heat exchanger.

6. The method according to claim 5, wherein the percolation fluid is heated to temperature of 34 to 45° C., preferably 35 to 42° C. before recirculating it to the biomass.

7. An arrangement for producing biogas, comprising:
a) a biomass reactor;
b) a fluid reactor;
c) a fluid collector for recovering percolation fluid;
d) a device for recirculating the percolation fluid to the biomass; and
e) a device for recovering the biogas;
wherein the device for recirculation of the percolation fluid is arranged to circulate an upper fraction of the fluid in the fluid reactor to upper section of the biomass, and wherein an upper fraction of the percolation fluid refers to the upper third of the percolation fluid.

8. The arrangement according to claim 7, wherein the arrangement is essentially gas tight.

9. The arrangement according to claim 7, further comprising a filler material for immobilization of microbes in the fluid reactor.

10. The arrangement according to claim 7, further comprising a heat-exchanger configured to heat the percolation fluid of item d.
